(19) United States
(12) Patent Application Publication
(10) Pub. No.: US 2022/0370003 A1
(43) Pub. Date: Nov. 24, 2022

(54) Transluminal Device and Method for the Mechanical Characterisation of Structures

(71) Applicant: Universidad de Granada, Granada (ES)

(72) Inventors: Guillermo RUS CALBORG, Granada (ES); Nader SAFFARI, Granada (ES); Antonio GOMEZ FERNANDEZ, Granada (ES); Elena SANCHESZ MUNOZ, Granada (ES)

(21) Appl. No.: 17/602,400

(22) PCT Filed: Mar. 26, 2018
(86) PCT No.: PCT/ES2018/070243

§ 371 Date: Oct. 8, 2021

(30) Foreign Application Priority Data
Mar. 24, 2017 (ES) P201730415

(51) Int. Cl. A61B 5/00; B06B 1/06

(52) U.S. Cl. CPC A61B 5/4381; A61B 5/0051; A61B 5/6852; B06B 1/0611; A61B 2018/0088

(57) ABSTRACT

The invention describes a device comprising at least one emitter of P-waves and/or S-waves, preferably shear waves, more preferably axisymmetric waves, and at least one wave receiver, wherein the receiver or receivers are disposed concentrically, and the disposition of the emitters and receivers allows same to simultaneously come into direct contact with a specimen, the structure of which it is desired to characterise. Also described is a method for characterising the spatial distribution of mechanical parameters of a specimen, based on the emission of shear waves and the subsequent reception thereof.

### TECHNICAL FIELD

The present invention relates to piezoelectric transducers widely used in the industries dedicated to medical diagnosis and industrial and aeronautical monitoring, among others. The invention relates to a transluminal device for generating and receiving mechanical shear waves in soft solid media with a Poisson's ratio close to 0.5 (quasi-incompressible media) containing an accessible lumen, such as gels and other viscous fluids. Devices of this type allow to be obtained information about the elastic characteristics of the medium to be studied and their distribution throughout same. In certain situations, changes in consistency of the tissue may indicate the presence of certain pathologies, which would allow this technique to be used for diagnostic purposes. In turn, certain targeted thermal treatments generate irreversible transformations in the consistency of the tissue, so the progression thereof could be monitored.

The field of application of the invention ranges from the non-destructive analysis and mechanical characterisation of materials, to quantitative dynamic elastography of biological tissues. In particular, this invention is applicable for performing transurethral elastography analysis for the diagnosis of prostate cancer and monitoring thermal ablation as a targeted therapy for prostate cancer.

### STATE OF THE ART

Shear mechanical waves, also known as shear waves, applied in a transluminal manner in a specific segment of the luminal wall, form a quasi-spherical distribution of shear waves which are propagated from the area of application in the wall of the lumen into the medium. Their behaviour is basically governed by the shear viscoelastic parameters of the medium.

The propagation of shear waves is governed by the shear mechanical parameters of the medium to be studied. In the case of longitudinal waves, the volumetric compressibility parameters are what govern their behaviour. In the case of the first application of the invention, that is, prostate imaging, as well as in most soft tissues, compressibility parameters vary by mere fractions of a percentage, whereas shear parameters vary by several orders of magnitude. This allows for the use of imaging techniques based on shear wave transducers which can detect alterations in the elasticity of the medium where the techniques based on compression wave transducers do not allow it. A clear example of this phenomenon is stiff prostate tumour imaging.

A transducer is a device capable of transforming or converting a specific type of input energy into another different energy at the output. Included among these devices are electromechanical transducers, which transform electrical energy into mechanical energy in the form of displacements elastically coupled with stress, in a bidirectional manner. Piezoelectric transducers are a type of electromechanical transducer, which emit and receive mechanical waves allowing ultrasound and/or elastographic imaging applications.

Torsion-based shear wave generators with applications in geophysics are known. This is the case of U.S. Pat. No. 5,321,333, which has a bilateral device (it generates respective waves at each end) for generating shear movements based on the combination of polarised piezoelectric elements, which are attached to a solid rod for transmitting the movement. However, applications in geophysics, unlike those in biomedicine, use very low frequencies given that the elements to be monitored are on the metre scale.

Transducers which emit torsion waves from accessible surfaces with applications for elastography in soft tissues, such as those described in WO 2012172136, are also known. In this patent, the generation of torsion waves is performed as a result of a transmission disc which combines a pair of elastic discs which provide the inertia needed to reduce the resonant frequency and stiffness to reduce dilatational waves, and a selection of transversely polarised piezoelectric elements which transform the electrical signal into mechanical movement. Nevertheless, the signal received with the described devices contains too much noise, so the analysis thereof presents serious difficulties. The lack of quality of this signal does not allow a correct reconstruction of the mechanical characteristics of the medium in specific situations.

Application WO/2017/009516 describes an electromechanical device which allows axisymmetric waves to be emitted and contacts the tissue on a face perpendicular to the axis of rotation of the contact element.

Vascular elastography devices such as the one described in US 20070282202 A1 are also known. This patent described in detail the design and use of a system for vascular strain elastography. This type of elastography is based on the comparison of the radio frequency obtained signals before and after applying compressive strain to the tissue to be studied. The result is an elasticity contrast map, but the map is lacking quantitative information about the mechanical parameters of the tissue.

There are no known transluminal devices with applications in quantitative dynamic elastography as of the date of this document.

In the specific case of the first application of this invention, different types of elastography for prostate applications can be found, but none of them is performed through the urethra.

Prostate Elastography

Prostate elastography is an emerging modality of medical imaging which consists of evaluating the stiffness of the prostate. Similarly to the wound healing process, it is thought that normal tissue stroma responds in an effort to repair the damage caused by the invasion of cancer cells. Said reaction is characterised by a high deposition of collagen. Given that the increase in the deposition of collagen entails an increase in the stiffness of the cancerous tissue, it has been suggested in a number of studies that the quantitative estimate of tissue stiffness can be an effective biomarker for evaluating the degree of prostate cancer and identifying the most aggressive tumours.

Real-time elastography is available in some ultrasound systems for prostate imaging together with other techniques that are currently being developed. There are two different elastography approaches commercially available today:
Strain elastography (SE).
Dynamic elastography, primarily by means of acoustic radiation force (ARF).

Strain elastography (SE) in the prostate is based on the comparative analysis of the strain of tissue before and after applying slight mechanical compression through the wall of the rectum. The stiffest zones experience less strain than those less stiff zones. Relative changes in the degree of strain provide an idea about zones with a higher stiffness and therefore suspected of containing pathogenic nodules. Quantitative stiffness values are not provided. This technique is commercially available on several medical ultrasound platforms. It does present some limitations:
Lack of non-uniform compression on the entire prostate, which can generate false interpretations.
Intra-e inter-operative dependence.
Difficulty to penetrate larger sized prostates.
Artifacts due to slippage on the compression plane.

Dynamic elastography for the detection of prostate cancer has mainly been tested using shear wave elastography (SWE) transrectally by means of Aixplorer ultrasound equipment (SuperSonic Imagine, Aix-en-Provence, France). In transrectal SWE, the ARF generates a shear wave front with a conical geometry with a small slope. This wave propagates inside the tissue from the zone of ARF generation outwards. An ultrafast ultrasound scanner allows the real-time wave propagation tracking, thereby obtaining its propagation speed and therefore an elasticity map of the tissue. The spatial resolution is worse than the resolution generated by SE, but quantitative stiffness values are provided.

Recent studies on the diagnosis of prostate cancer using SWE transrectally have shown very promising results. The use of a threshold Young's modulus between lesions and normal tissue of 35 kPa in the peripheral zone of the prostate can provide further information for the detection of cancer and the guidance of biopsies, allowing a substantial reduction in the final number of biopsies to be performed. The limitations of this technique are:
Pressure artifacts due to the design of the transrectal transducer, which requires tilting the catheter to scan the mid prostate and its apex.
Slow acquisition of images, namely one image per second.
Limited size of the region of interest, namely only a 2D plane of half of the prostate is covered.
Lag in the signal stabilisation time for each acquisition plane.
Attenuation of the signal in prostates of large dimensions, which hinders the evaluation of the anterior zone of the prostate.

The reason that there is no immediate plan to design a transluminal ultrasonic catheter with the capacity to generate sufficient acoustic radiation force that it generates a shear wave being propagated inside the tissue is primarily the lack of space for containing the size of an acoustic lens that this technique requires. At the same time, a lack of space for incorporating the shear wave propagation monitoring system would also be observed. It is for this reason that it is necessary to conceive of a design where the shear wave is generated by mechanical actuators, and where the detection system allows the miniaturisation thereof.

Mechanical Characterisation of Structures

The physical principle for mechanically characterising the structure of a medium is the following: A physical magnitude is propagated in the form of a wave through the medium to be analysed, which distorts the wave until it is measured on an accessible surface. The mechanical parameters responsible for the modification of the wave can be deduced from of the measurements performed by means of the model-based theory of inverse problems. This technique is the strongest known strategy for now.

The use of genetic algorithms for cost function optimisation, the variables of which are the mechanical parameters to be quantified, has previously been described. Other complementary methods such as reverse time migration can help to previously reduce the size of the optimisation domain of the genetic algorithm.

### OBJECT OF THE INVENTION

The object of the invention relates to a piezo-electromechanical transducer catheter containing a set of shear wave transmitters and receivers for solid, quasi-incompressible media and some fluid gels, for transmitting and receiving shear waves from a cavity inside the medium, or by inserting the catheter through an accessible surface in the case of fluid gels. The rotational oscillating force induced by each emitter is transmitted in the medium in the form of pseudo-spherical radiations of shear waves. Analysis of the waves detected by the receivers, once they have travelled through the tissue, allows obtaining valuable information about the elastic state and spatial distribution thereof, which would allow, for example, the detection of zones of greater stiffness which may be associated with tumours.

All the existing prostate elastography methods are based on the transrectal approach. In the case of this invention, access to the gland would be transurethral, which involves a series of inherent advantages:
Better accessibility to the anterior zone of the prostate, which is less accessible when transrectal access is used.
Possibility of using frequencies (greater than 500 Hz) that are higher than those used at present. This would allow greater spatial resolution and therefore a better detectability of tumours of small dimensions.
Ability to scan the entire gland in a single process, thereby obtaining a 3D mechanical parameter map of the tissue.
Low thermal and mechanical levels compared with techniques that use ARF as a shear wave excitation source.
Use of the urethra as an access channel keeps the rectal passage free for transrectal therapies such as HIFU thermal ablation, which would allow such therapies to be monitored.

Therefore, a first aspect of the invention consists of a transluminal or intraluminal catheter for analysing the structure of a specimen comprising at least one emitter of S-waves or of P-waves and S-waves, preferably shear waves, more preferably axisymmetric waves, and at least one wave receiver, wherein the receiver or receivers are disposed concentrically, and the disposition of the emitters and receivers allows same to simultaneously come into direct contact with the specimen.

In a second aspect, the invention relates to a method for obtaining data useful for characterising the spatial distribution of mechanical parameters of a specimen, in particular the elastographic analysis of the specimen, preferably obtaining parameters useful as biomarkers, comprising the emission of P-waves and/or S-waves, preferably shear waves, more preferably axisymmetric waves, and the extraction of mechanical constants from the reception of waves reflected from a catheter positioned inside a vessel or conduit of the specimen.

### DESCRIPTION OF THE FIGURES

FIG. 1 shows a depiction of two concentric dispositions of the receivers of the catheter (S), of the invention where ® represents a receiver and (d) the distance from the outer surface of the receiver to the longitudinal axis, (0).

FIG. 2 shows a schematic depiction of an emitter formed by a contact element (C) with a noticeably toroidal shape attached at its inner part to four piezoelectric elements (Pz). (0) represents the longitudinal axis of the catheter of the invention, (T) the direction of movement of the contact element and (E) a central element on which the different elements forming the zone are fixed.

FIG. 3 shows a schematic depiction of an emitter or receiver formed by a contact element (C) with a noticeably toroidal shape attached at its inner part to four piezoelectric elements (Pz). (0) represents the longitudinal axis of the catheter of the invention, (T) the direction of movement of the contact element and E a central element on which the different elements forming the zone are fixed.

FIG. 4 shows a schematic depiction of two sets of 3 and 4 receivers formed by contact elements (C) with a toroid segment shape, the axis of revolution of which coincides with the centre on the longitudinal axis (0), and on the inner part of which there is fixed a piezoelectric element (Pz) with a polarisation which allows a rotational movement with a tangential direction (T) relative to the outer surface of the contact element to be transformed into an electrical signal. E represents a central element on which the different elements forming the zone are fixed.

FIG. 5 shows a diagram of a particular embodiment of the device with 5 sets of 4 receivers (j=5, k=4). (0) represents the longitudinal axis of the catheter of the invention and (E) a central element on which the different elements forming the zone are fixed.

FIG. 6 shows a schematic depiction of the position of the catheter inside a vessel (V) of a specimen (Sp).

FIG. 7 shows a schematic depiction of the position of the catheter inside a vessel (V) of a specimen (Sp) before and after suctioning out the air.

FIG. 8 shows a diagram by way of an embodiment in which the catheter comprises four sets of 4 receivers (receivers) and a wave emitter (emitter) with a toroidal shape.

FIG. 9 shows a diagram by way of an embodiment in which the catheter comprises four receivers (receivers) and a wave emitter (emitter) with a disc shape and is connected to an electromagnetic motor (Motor).

FIG. 10 shows experimental signals obtained with the simulated prostate prototype with 13% gel.

FIG. 11 shows simulated signals for the simulated prostate prototype with 13% gel.

FIG. 12 shows a depiction of a simulated prostate. (a) depicts the diagram of the 2D shear wave propagation models. (b) details the geometry and dimensions of the simulated prostate used for the validation experiments.

FIG. 13 shows a diagram of the system used for carrying out the optical experiments to capture the displacement of particles embedded in the simulated prostate.

FIG. 14 shows a comparative example between two scenarios, one simulated (a) and the other one obtained from optical experiments (b), of the propagation of the shear wave generated by the wave emitter of the invention while it is propagated from the conduit simulating the urethra (coordinate r=5.5 mm) to the area where the inclusion-tumour is located (coordinate r=15 mm).

FIG. 15 shows a comparative example between amplitude spectra of two scenarios, one simulated (a) and the other one obtained from optical experiments (b), along the propagation path of the shear wave going from the wave emitter to the area where the inclusion-tumour is located.

### DETAILED DESCRIPTION OF THE INVENTION

### Definitions

Throughout the present specification, “axisymmetric shear wave” or “ASW” will be understood to mean a mechanical shear wave propagated in quasi-incompressible media, preferably biological tissues, governed by the deflection component of elasticity and propagated at the speed of shear waves, in the radial and axial directions, according to the mathematical model in a first approach described below.

The equations which describe the propagation of the axisymmetric wave, as well as the angular oscillatory displacement that the particles of the medium experience upon the passage of the wave can be described by the equations for the conservation of momentum, for the equilibrium between strains and displacements, and lastly mechanical constitutive equations of the propagation medium. This last group of equations describes how the propagation medium responds in terms of strain when subjected to stresses; a number of constitutive models have been proposed. This document sets forth the so-called Kelvin-Voigt Fractional Derivative (KVFD), as it is a generalisation of other simpler constitutive models, in addition to being postulated in recent literature as one of the ideal constitutive models for the simulation of dynamic elastography:

Conservation of Momentum:

$$\frac{{\partial }^{2} {u}_{θ}}{\partial {t}^{2}} = \frac{1}{ρ} ( \frac{\partial {σ}_{r θ}}{\partial r} + \frac{\partial {σ}_{θ z}}{\partial z} + \frac{2}{r} {σ}_{r θ} )$$

Where u_{θ} is the angular displacement of the particles, ρ is the density of the propagation medium, t is the time, σ_{rθ} and σ_{θz} are the shear stresses, r is the radial coordinate and z is the axial coordinate.

Equilibrium between strains and displacements:

$${ε}_{r θ} = \frac{1}{2} ( \frac{\partial {u}_{θ}}{\partial r} - \frac{{u}_{θ}}{r} ) ;$$

$${ε}_{θ z} = \frac{1}{2} \frac{\partial {u}_{θ}}{\partial z}$$

Where ε_{rθ} and ε_{θz} are shear strains.

KVFD Constitutive Model:

$${σ}_{r θ} = 2 μ {ε}_{r θ} + 2 η \frac{{\partial }^{α} {ε}_{r θ}}{\partial {t}^{α}} ; 0 < α < 2$$

$${σ}_{θ z} = 2 μ {ε}_{θ z} + 2 η \frac{{\partial }^{α} {ε}_{θ z}}{\partial {t}^{α}} ; 0 < α < 2$$

Where μ is the instantaneous elastic behaviour component of the propagation medium, η is the viscous behaviour component of the propagation medium, and α is the fractional derivative order, also related to the law of power which describes wave attenuation depending on the frequency thereof.

A device is said to be “intraluminal” or “transluminal” when it is suitable for being introduced into a vessel or conduit. A urinary line or catheter are considered transluminal devices.

“Toroid” will be understood to mean the surface of revolution generated by a simple, closed flat curve or a polygon which turns around a coplanar outer line (axis of rotation) with which it does not intersect.

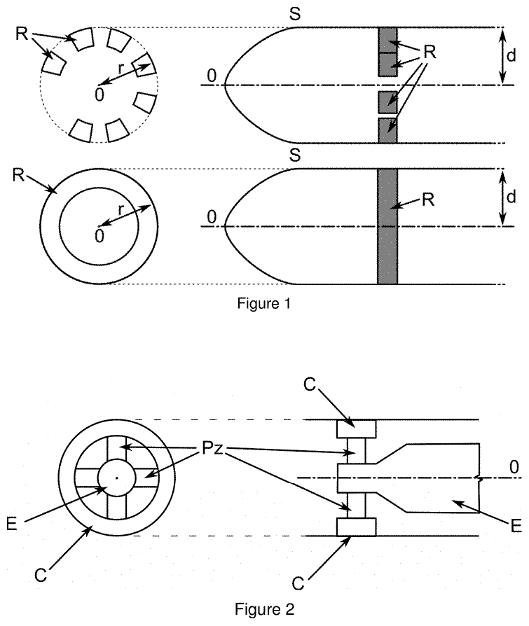

A plurality of emitters or wave receivers positioned in an intraluminal device, S, are said to be “disposed concentrically” or “disposed equidistantly relative to the longitudinal axis” [FIG. 1], when the distance, d, of the outer surface of the emitters or receivers, R, to the longitudinal axis of the device, 0, is constant. By extension, this definition also includes the case in which an emitter or receiver has a cylindrical or toroidal shape and its axis of revolution coincides with the axis of the device.

Throughout the present description, “specimen” will be understood to mean the material, preferably tissue, more preferably live tissues, through which the waves emitted by the transducer are passed in order to know their structural characteristics (elastic parameters, viscoelastic parameters, parameters of the microstructural geometry, porous geometry, or energy dissipation models, among others).

For purposes of the present invention, “electromechanical actuator” will be understood to mean a device capable of transforming electrical energy into a movement, particularly a rotational movement. In a particular embodiment suitable for this invention, the electromechanical actuator is stimulated with an electrical signal generated by an electric pulse generator and is capable of transforming that signal into a minimum fraction of a turn, which will serve to generate the wave that is subsequently analysed. In a more particular embodiment, the electromechanical actuator will be an electromagnetic motor, such that the rotation is induced by transforming electrical energy into magnetic energy.

An example of actuators of this type can consist of a small-sized electromagnetic motor or micromotor.

For purposes of the present invention, the electromechanical actuator is stimulated by means capable of generating electrical signals or waves, hereinafter “electrical signal generator”. “Electrical signal” will be understood to mean electrical magnitude, the value of which depends on time. For purposes of the present invention, constant magnitudes will be considered particular cases of electrical signals.

The electrical signals generated by an electrical signal generator can be periodic (sine, square, triangular, “saw-tooth” shaped, etc.). In this manner, when connected to an actuator which transforms the signal into a rotational movement, the actuator turns a minimum fraction of a turn depending on the voltage, frequency and/or time between pulses that are determined by the signal. Any electronic circuit which digitises the electrical signals at the desired frequencies can be used as an electrical signal generator. Another example of an electrical signal generator used in the experimental designs of the present invention can be an oscilloscope, since it allows an electrical signal with a voltage that varies over a specific time to be emitted.

“Biocompatible material” will be understood to mean a material with the composition of which neither interferes with nor degrades the biological medium in which it is used. These materials are normally used to make devices or elements thereof which must be in either brief or prolonged direct contact with the internal fluids and tissues of the body, such as catheters, syringes, prostheses, etc. An example of this material is the polylactic acid (PLA).

“Contact element” shall refer to the part or element positioned in the distal or anterior part of the emitter or receiver and contacting the specimen on which the wave is intended to be transmitted. Preferably, the surface of the contact element that contacts with the specimen must approximate the curvature of the section of the lumen so as to allow suitable transmission of the wave. Also preferably, the contact element of an emitter or receiver will be manufactured in a material with a shear acoustic impedance comprised between the shear acoustic impedance of the piezoelectric elements and the shear acoustic impedance of the specimen for the purpose of maximising the energy of the waves that will be emitted on same.

“Shear acoustic impedance” shall refer to the value zₛ determined by the equation

zₛ=cₛρ

Where zₛ is the shear acoustic impedance in a specific volume of the propagation medium, ρ is the density, and cₛ is the speed of the shear wave in the same specific volume of the propagation medium.

Catheter of the Invention

In the defined context, a first aspect of the invention relates to a transluminal or intraluminal catheter for characterising the spatial distribution of mechanical parameters of a specimen, hereinafter “the device or the catheter of the invention”, comprising at least one emitter of S-waves or of P-waves and S-waves, preferably shear waves, more preferably axisymmetric waves, hereinafter “wave emitter”, and at least one wave receiver, wherein the receiver or receivers are disposed concentrically, and the disposition of the emitters and receivers allows same to simultaneously come into direct contact with the specimen.

In a particular manner, the device of the invention can generate axisymmetric waves at different frequencies controlling electrical excitation. The device can generate waves comprising frequencies ranging from 1 Hz to 50 MHz depending on the dimensions and the materials of the specimen.

In a particular embodiment, the catheter of the invention comprises at least one concentrically positioned wave emitter. In a preferred embodiment, the catheter comprises a single concentrically positioned wave emitter.

In another particular embodiment, at least one wave emitter, preferably each wave emitter, comprises a contact element attached to an electromechanical actuator.

In a preferred embodiment, at least one wave emitter, preferably each emitter, comprises a disc- or cylindrical-shaped contact element attached to an electromagnetic device converting electrical signals into rotational movement, such as an electromagnetic micromotor. An example of emitters of this type can be found in application WO/2017/009516.

In a preferred embodiment in which the catheter of the invention comprises a wave emitter in turn comprising a disc- or cylindrical-shaped contact element attached to an electromagnetic device capable of converting electrical energy into a rotational movement, the attachment of the contact element to the device which provides the rotational movement is performed by means of a flexible shaft with a length greater than 5 cm, preferably greater than 25 cm and more preferred greater than 30 cm, which translates the induced rotational movement, allowing said electromagnetic device to be positioned outside the conduit or vessel in which the distal part of the catheter is introduced and allowing the diameter thereof to be reduced.

In another particular embodiment [FIG. 2], at least one wave emitter comprises at least one piezoelectric element (Pz), preferably two or more piezoelectric elements, fixed to the inner part of a contact element (C) with a noticeably toroidal shape, the axis of revolution of which coincides with the centre on the longitudinal axis of the catheter (0) and the polarisation of the piezoelectric element or elements allows an electrical signal to be transformed into a rotational movement with a tangential direction (T) relative to the outer surface of the contact element.

In another particular embodiment, at least one wave receiver, preferably each receiver, comprises a contact element attached at least to one piezoelectric element such that when a wave reaches a receiver, the contact element resonates and deforms the piezoelectric elements, producing an elastic signal coupled with its stress state.

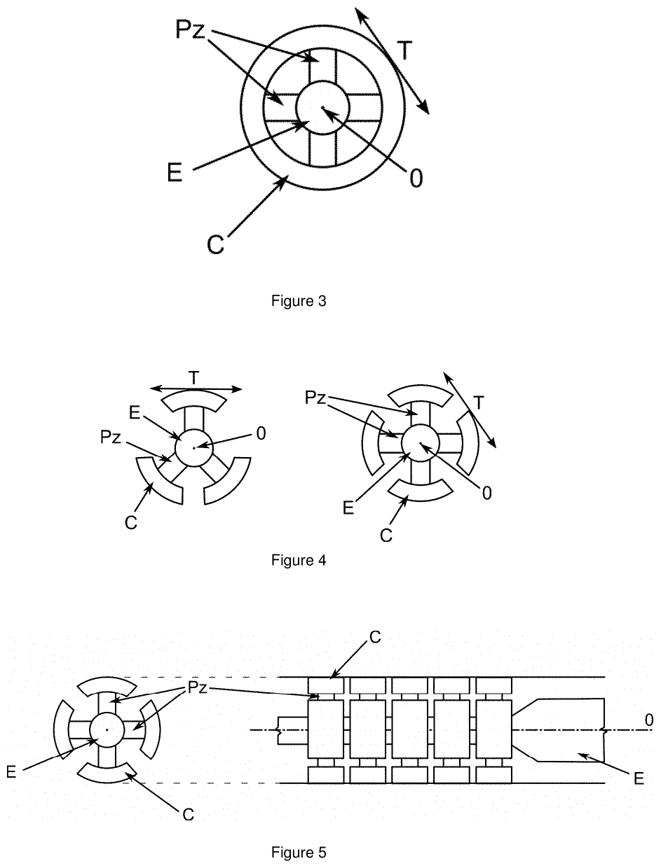

In another particular embodiment [FIG. 3], at least one receiver, preferably each receiver, comprises at least one piezoelectric element (Pz) fixed to the inner part of a contact element (C) with a noticeably toroidal shape, the axis of revolution of which coincides with the centre on the longitudinal axis (0) of the catheter, and the polarisation of the piezoelectric element or elements allows a rotational movement with a tangential direction (T) relative to the outer surface of the contact element to be transformed into an electrical signal.

Preferably [FIG. 4], the contact elements (C) of the wave receivers are segments of a cylinder or a toroid, the axis of revolution of which coincides with the centre on the longitudinal axis (0), and on the inner part of which there is fixed a piezoelectric element (Pz) with a polarisation which allows a rotational movement with a tangential direction (T) relative to the outer surface of the contact element to be transformed into an electrical signal. A set of k receivers, with k≥1, preferably k≥2, more preferably k=4, is thereby disposed, positioned in the same position of the longitudinal axis of the catheter. By increasing the number k of receivers, the catheter is made to be more sensitive to the three-dimensional heterogeneity of the specimen.

In another particular embodiment, the catheter of the invention comprises a single wave emitter positioned between the contact element and the receivers. In a preferred embodiment, the single emitter comprises a contact element with cylindrical shape attached to an electromagnetic device which provides the rotational movement and is positioned between the contact element and the receivers.

In another particular embodiment, the contact element of at least one wave emitter, preferably the contact element of each emitter, has a cylindrical shape and presents a plurality of holes which facilitate the air existing between the catheter and the wall of the vessel or conduit to be suctioned out, such that there is no separation between the receivers and the tissue.

In a particular embodiment, the catheter of the invention comprises at least 2, preferably at least 3, concentrically positioned receivers.

In another preferred embodiment, the catheter of the invention comprises j sets or blocks of receivers, where j≥2, such that the j sets of receivers are positioned aligned along the longitudinal axis of the catheter [FIG. 5]. In a more preferred manner, the catheter of the invention comprises j sets of k receivers, where j≥2 and k≥2, more preferably j≥2 and k≥3 and even more preferred, j≥3 and k≥3,

In a more preferred manner, the contact elements of each set of receivers are segments of one and the same cylinder or of a toroid, the axis of revolution of which coincides with the centre on the longitudinal axis. In a specific embodiment, each of these segments is attached to a piezoelectric element such that all the piezoelectric elements of each set of receivers have one and the same polarisation in direction that is tangent to the outer surface of the cylinder or toroid.

Ideally both the emitters and the receivers have to be in contact with the specimen; therefore, in another particular embodiment, the catheter of the invention is coated with a protective layer, preferably manufactured with a hydrophobic material, preferably with a thickness of between 30 and 60 micra, such that the contact elements will be separated from the specimen only by this layer for hygiene purposes, without attenuating the signal or introducing interferences. Preferably, this protective layer will be disposable.

In another preferred embodiment, the device of the invention comprises means which allow the air existing between the surface of the catheter and the wall of the vessel or conduit to be suctioned out, such that there is no separation between the receivers and the tissue. By extension, these means for suctioning out the air can be part of another device that is used in an auxiliary manner to facilitate the elastographic analysis. An example of these means consists of a vacuum pump, a peristaltic pump or a syringe.

The catheter of the invention is completed with means suitable for transmitting the electrical signals which induce the movement of the emitters and receiving the signals picked up by the receivers, as well as means which allow the storage and processing of data obtained with the catheter.

Method for Obtaining Data Useful for the Elastographic Analysis

Therefore, the invention relates to a method, hereinafter “method of the invention” for obtaining data useful for characterising the spatial distribution of mechanical parameters of a specimen, in particular the elastographic analysis of the specimen, preferably obtaining parameters useful as biomarkers, comprising the emission of P-waves and/or S-waves, preferably shear waves, more preferably axisymmetric waves, and the extraction of mechanical constants from the reception of waves reflected from a catheter positioned inside a vessel or conduit of the specimen.

In a particular embodiment, the method of the invention emits and receives the waves by means of the catheter of the invention.

The extraction of constants or mechanical parameters, in particular parameters useful as biomarkers, which govern the propagation of the waves from the waveform over time recorded by the receiver can be performed through a mere time-of-flight calculation from the start of the signal over time, to an inverse problem based on models of propagation simulated by semi-analytical or numerical methods.

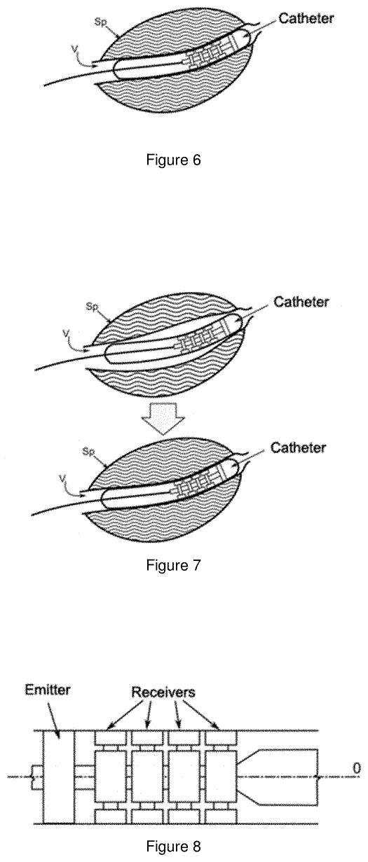

In a particular manner, the method consists of introducing a catheter capable of emitting axisymmetric waves and receiving the reflected waves, preferably the catheter of the invention, through a vessel or conduit, such as an artery, or preferably the urethra, until reaching the position closest to the zone of the specimen which is intended to be analysed, emitting shear waves, preferably axisymmetric, and collecting the signal of the reflected wave [FIG. 6].

In a particular embodiment, the method comprises a prior step in which the gas or fluid existing inside the conduit is suctioned out to maximise the contact surface between the walls of the conduit and the contact elements of the catheter of the invention [FIG. 7], thereby allowing better propagation of the emitted and received waves.

In a preferred embodiment, the method of the invention is a method for performing transurethral elastography analysis for the diagnosis of prostate cancer and monitoring thermal ablation as a targeted therapy for prostate cancer.

Elastographic Parameter Reconstruction Method

The elastographic parameters can be reconstructed from the signals received by the receivers of the catheter of the invention by means of any of the methods described in the state of the art as optimisation methods, such as genetic algorithms, or other types such as reverse-time migration, widely used in geophysics, which make use of a propagation model, such that the closer the model is to reality, the more reliable the reconstruction of the parameters of the lesions will be. In a preferred embodiment, the propagation model used is the KVFD constitutive model.

In a particular embodiment, the movement induced by each emitter can be broken down into a pseudo-spherical shear wave source, minimising the emission of compression waves. A genetic algorithm which optimises a cost function the variables of which are the mechanical parameters to be quantified is subsequently used. Other complementary methods such as reverse-time migration can help to previously reduce the size of the optimisation domain of the genetic algorithm.

The physical principle is based on the interaction between the shear waves transmitted and the internal mechanical structure of the specimen of interest. The application for the diagnosis of prostate cancer and the monitoring of targeted prostate ablation can be mentioned as examples. The shear waves propagated in the prostate tissue will be altered by the presence of lesions that are stiffer than the surrounding tissue. Both the majority of prostate tumours and the tissue treated by thermal ablation have a high stiffness in reference to normal prostate tissue. These elastic changes in the tissue generate wave reflections, which are detected by the catheter, and which, as a result of the treatment thereof and the application of inversion methods, parameters of those lesions can be reconstructed, such as the elastic modulus, viscosity, size and location.

### Embodiment of the Invention

### Examples of Catheters

In a first embodiment, the catheter of the invention comprises:
A single emitter of axisymmetric waves [FIG. 4] formed by a contact element (C) manufactured in PLA and with a torus shape with a square section attached to 4 piezoelectric elements (Pz) fixed at their inner part in equidistant positions, such that the rotational movement (T) of the contact element is induced by said piezoelectric elements.
A set of 4 receivers [FIG. 5], each one formed by a contact element (C), the shape of which is an 80° section of a PLA cylinder 1 mm thick and attached to a piezoelectric element (Pz) at its inner part.

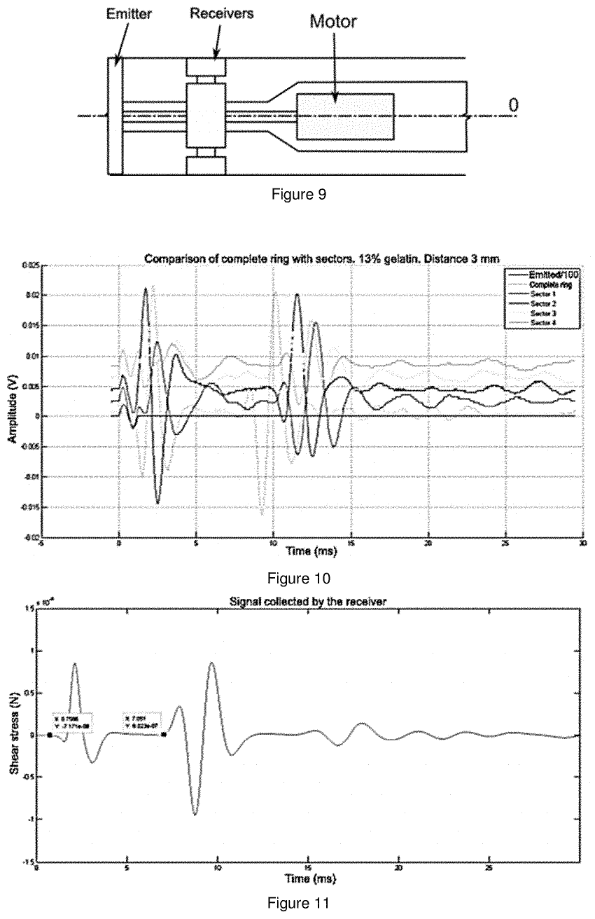

In a second embodiment [FIG. 8], the reception of these waves is achieved by means of 4 sets of receivers positioned equidistantly along the longitudinal axis of the catheter (0), wherein each set is made up of 4 receivers positioned with an identical angle between every two, wherein each receiver is formed by a contact element (C), the shape of which is an 80° section of a PLA cylinder 1 mm thick and attached to a piezoelectric element (Pz) at its inner part.

In a third embodiment [FIG. 9], the catheter of the invention comprises:
A single emitter of axisymmetric waves formed by a contact element manufactured in PLA and with a disc shape attached to an electromagnetic motor connected to an oscilloscope.
A set of 4 receivers [FIG. 5], each one formed by a contact element (C), the shape of which is an 80° section of a PLA cylinder 1 mm thick and attached to a piezoelectric element (Pz) at its inner part.

The catheters described as examples is completed with electronics capable of exciting the emitter and conditioning and digitising the reception, the software capable of implementing the operating and analysis interface, as well as the structure and casing capable of housing the previous elements under conditions of hygiene and ergonomics.

### Experimental Results 1

Using the prototype described in the third embodiment, with a receiver comprising a contact element formed 4 ring sectors attached to 4 piezoelectric elements and a single electromechanical wave emitter with an emitter designed in the form of a disc, on a simulated prostate consisting of 13% bovine origin gelatin (Sigma Aldrich, Bovine skin gelatine 225 bloom, type B) in water, the following results have been obtained from measured signals [FIG. 10], which allows the prototype to be validated against simulated signals by means of finite differences using the theory of linear elasticity simulating the same configuration [FIG. 11], and are compatible with the experimental results, thereby validating on a preliminary basis prototype functionality.

### Experimental Results 2

Again using the wave emitter of the prototype described in the third embodiment, a series of experimental tests have been performed in a medium with viscoelastic properties similar to the human prostate (simulated prostate), which have been used to verify that the propagation of shear waves and the reflection produced when the shear wave front reaches a region with change in viscoelastic properties, are generated in a satisfactory manner by the wave emitter of the invention, and that the propagation model based on a constitutive KVFD law truthfully simulates the physical phenomenon of propagation, the attenuation of the waves and the interaction with changes in the distribution of the viscoelastic properties of the prostate, which confirms the viability of the method of the invention.

General Description of the Simulated Prostate

Several simulated prostates were designed and manufactured to experimentally observe the propagation of shear waves generated by the wave emitter disc of the catheter described as an example in the third embodiment, and the interaction with areas of mayor stiffness, as occurs with most prostate tumours.

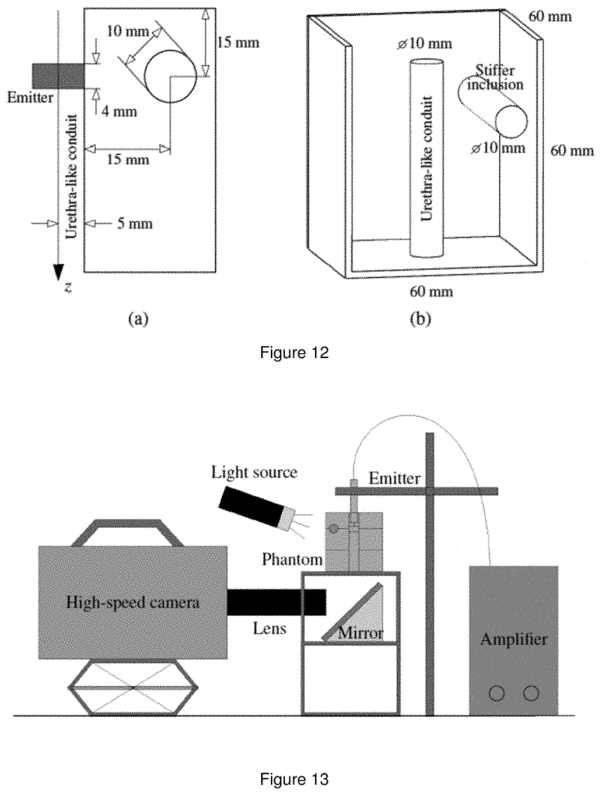

The geometry of the simulated prostate is the result of extending to three dimensions (3D) the two-dimensional propagation model (2D) included in the reconstruction methods (FIG. 12). Although the simulated prostate has a cubic geometry and, therefore, is not an exact reproduction of the anatomy of the prostate, it has been considered valid for the purpose of the experimental study. Additionally, the chosen dimensions, both external dimensions and the dimensions of the diameter of the urethra and of the inclusion-tumour, are consistent with the characteristic dimensions that can be found in real cases.

To imitate the viscoelastic properties, a combination of bovine origin gelatin (Sigma Aldrich, Bovine skin gelatine 225 bloom, type B), formaldehyde as a cross-linking agent and potassium sorbate for delaying the proliferation of bacteria and fungi, was chosen. Different gelatin solutions with a different concentration of gelatin were used to manufacturing the backing material of the simulated prostate and the inclusions-tumours, specifically, the backing material was manufactured with a concentration of 9% of gelatin (weight/weight), whereas concentrations of 12, 14 and 17% were used to simulate tumours.

The viscoelastic parameters (complex shear modulus, where the real part is related to the elastic component and the imaginary part with the viscous component) of the different gelatin solutions were characterised using a conventional rheometer, thereby obtaining ratios of the shear modulus (absolute value of the complex shear modulus) between inclusion-tumour and backing material of 1.20, 1.70 and 3.80 for the inclusions of 12, 14 and 17% of gelatin, respectively.

Optical Experiments

Several sets of optical experiments using a super slow motion camera to capture the particle displacement generated by shear waves generated by the wave emitter of the prototype described in the third embodiment which are propagated through simulated prostates and interact with the rigid inclusion simulating a prostate tumour (FIG. 13) were designed and performed.

The simulated prostates were manufactured so as to be translucent, containing therein embedded basalt particles, such that by using an artificial illumination system and by means of the use of the super slow motion camera, observation of the basalt particle displacement upon passage of the shear waves was possible. Monocyclic signals with frequencies between 100 and 700 Hz were used.

By means of an algorithm based on cross-correlation, basalt particle displacements were reconstructed from the videos obtained by the super slow motion camera.

The phase speed of the shear waves for each frequency used in the optical experiments was calculated from the reconstructions of the particle displacements. Moreover, the values of the complex shear modulus obtained from experiments with a rheometer were used to calculate the theoretical phase speed of the shear waves for lower frequencies (close to the static case). This data relating to speed in the entire complete range of frequencies was used to estimate the viscoelastic parameters according to the KVFD constitutive model of the different areas of the simulated prostates, and to thus generate scenarios in the 2D wave propagation model which would be compared with the results of the optical experiments.

Results of the Validation

The scenarios simulated using 2D shear wave propagation models offered a truthful reproduction of the observations obtained from the optical experiments with a super slow motion camera in simulated prostates.

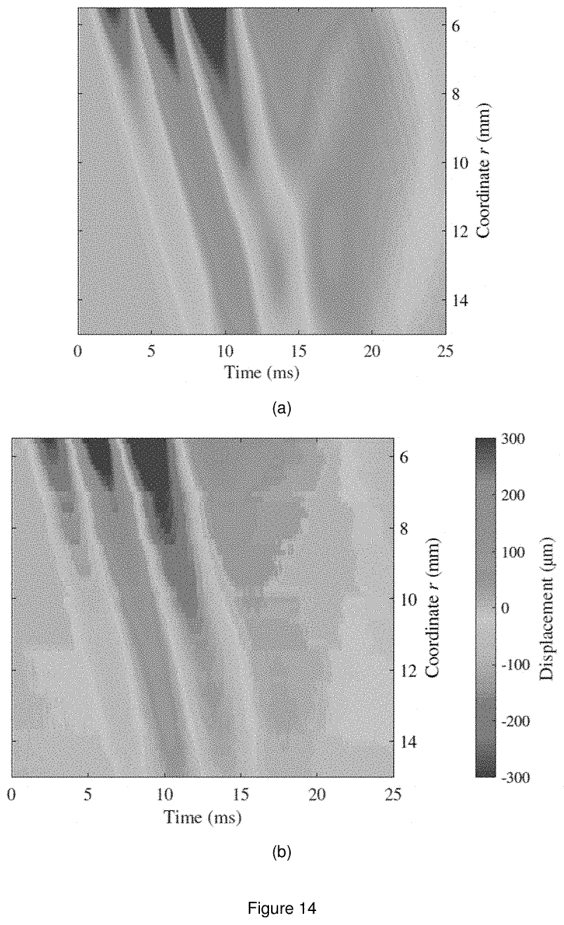

The FIG. 14 shows a comparative example between two scenarios, one simulated and another one obtained from optical experiments. The model truthfully reproduces space-time evolution of the shear wave from its generation in the area around the wave emitter until the wave front reaches the area where the inclusion-tumour is located. Likewise, the model shows a clear perturbation at times after the encounter with the inclusion-tumour, which perturbation consists of the reflection of the wave front which returns again to the area where the wave emitter is located. In a more general manner, the similarity between space-time patterns generated by means of the model and those obtained experimentally on the propagation of shear waves in simulated prostates was measured by means of the Pearson correlation coefficient, obtaining a high degree of correlation between propagation graphs, namely rPearson=0.9786±0.0068 (mean and standard deviation of all the compared space-time pattern pairs), thereby confirming the existence of shear waves generated by the wave emitter of the invention, and validating the working hypothesis of the generation of reflections which will allow the reconstruction of the location and parameters viscoelastic of tumours or other rigid inclusions located inside prostate glands.

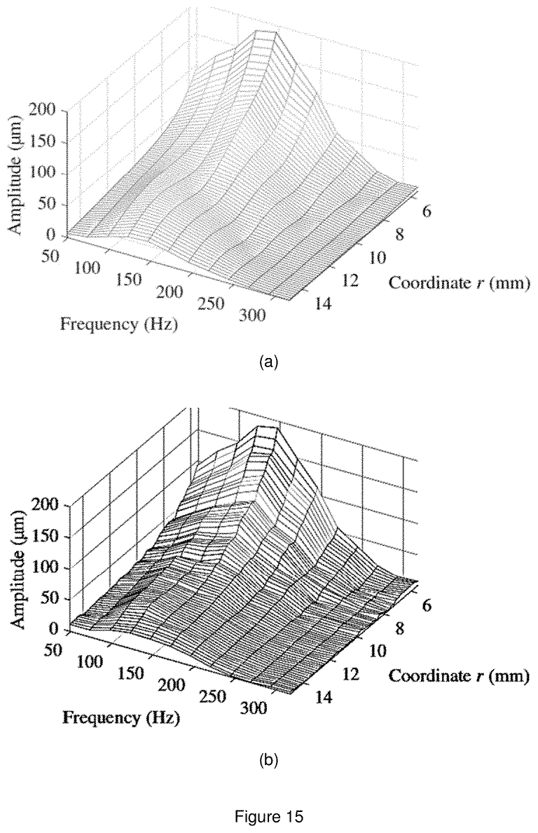

Finally, to completely validate the shear wave propagation model and verify that it correctly simulates the attenuation sustained by the shear waves upon their passage through the simulated prostates, a comparative study of the amplitude spectra obtained from the simulated scenarios and from the measurements obtained from the optical experiments was performed. The amplitude spectrum along the wave propagation path offers the possibility of comparing the attenuation predicted by the model and the attenuation observed in the optical experiments. FIG. 15 shows an illustrative example of this comparison, where both spectra coming from the experimental scenario and the simulated scenario offer very similar values and distributions. In a more general manner, the similarity between all the generated pairs of amplitude spectra coming from simulations with the 2D model and from experimental observations was calculated by means of the root-mean-square error (RMSE), obtaining a value of 3.36±1.20% (mean and standard deviation of all the compared pairs of amplitude spectra). Correct simulation of the propagation of shear waves in a situation similar to the one that will occur in the human prostate on the part of the 2D wave propagation model is thereby confirmed; therefore, the model could be used by the reconstruction methods for the location and characterisation of mechanical properties of tumours and other rigid lesions in the prostate.

### CONCLUSIONS

The propagation of shear waves generated by the wave emitter of the invention in media simulating the viscoelastic behaviour of prostate tissue has been observed experimentally. Likewise, interaction between waves of this type and areas of greater stiffness, as is the case of most prostate tumours and lesions as a result of targeted thermal ablation treatments in the prostate. Additionally, the 2D wave propagation model based on a KVFD constitutive law has been validated against experimental observations, which assures the use thereof in the reconstruction methods considered for obtaining the location and viscoelastic parameters of tumours or other rigid lesions in prostate tissue.

1. A transluminal or intraluminal catheter for characterising the spatial distribution of mechanical parameters of a specimen, comprising at least one emitter of S-waves or of P-waves and S-waves, and at least one wave receiver, wherein the receiver or receivers are disposed concentrically, and the disposition of the emitters and receivers allows same to simultaneously come into direct contact with the specimen.

2. The catheter according to claim 1, further comprising at least one emitter of shear waves.

3. The catheter according to claim 2, wherein at least one wave emitter is positioned concentrically.

4. The catheter according to claim 3, wherein the catheter comprises a single wave emitter.

5. The catheter according to claim 1, wherein at least one emitter of axisymmetric waves comprises a disc- or cylindrical-shaped contact element attached to an electromagnetic device converting electrical signals into rotational movement.

6. The catheter according to claim 5, wherein the attachment of the contact element to the device which provides the rotational movement is performed by means of a flexible shaft with a length greater than 5 cm, which translates the induced rotational movement.

7. The catheter according to claim 1, wherein the contact element of at least one emitter of axisymmetric waves has a cylindrical shape and presents a plurality of holes.

8. The catheter according to claim 1, wherein at least one emitter comprises at least one piezoelectric element fixed to the inner part of a contact element with a noticeably toroidal shape, the axis of revolution of which coincides with the centre on the longitudinal axis of the catheter, and the polarisation of the piezoelectric element or elements allows an electrical signal to be transformed into a rotational movement with a tangential direction relative to the outer surface of the contact element.

9. The catheter according to claim 1, wherein at least one receiver comprises a contact element attached at least to one piezoelectric element such that when a wave reaches a receiver, the contact element resonates and deforms the piezoelectric elements, producing an elastic signal coupled with its stress state.

10. The catheter according to claim 9, wherein at least one receiver comprises at least one piezoelectric element fixed to the inner part of a contact element with a noticeably toroidal shape, the axis of revolution of which coincides with the centre on the longitudinal axis of the catheter, and the polarisation of the piezoelectric element or elements allows a rotational movement with a tangential direction relative to the outer surface of the contact element to be transformed into an electrical signal.

11. The catheter according to claim 9, wherein the contact elements of the wave receivers are segments of a cylinder or a toroid, the axis of revolution of which coincides with the centre on the longitudinal axis, and on the inner part of which there is fixed a piezoelectric element with a polarisation which allows a rotational movement with a tangential direction relative to the outer surface of the contact element to be transformed into an electrical signal.

12. The catheter according to claim 1, wherein the catheter comprises at least 2 concentrically positioned receivers.

13. The catheter according to claim 12, wherein the catheter comprises j sets or blocks of receivers, where j≥2, such that the j sets of receivers are positioned aligned along the longitudinal axis of the catheter.

14. The catheter according to claim 13, wherein the catheter comprises j sets of k receivers, where j≥2 and k≥2.

15. The catheter according to claim 1, further comprising means which allow the air existing between the surface of the catheter and the wall of the vessel or conduit to be suctioned out, such that there is no separation between the receivers and the specimen.

16. A method for obtaining data useful for characterising the spatial distribution of mechanical parameters of a specimen comprising the emission of S-waves or of P-waves and S-waves, and the reception of the waves reflected from a catheter positioned inside a vessel or conduit.

17. The method according to claim 16, wherein the emitted waves are shear waves.

18. The method according to claim 16, further comprising a prior step in which the gas or fluid existing inside the conduit is suctioned out to maximise the contact surface between the walls of the conduit and the catheter.

19. The method according to claim 16 further comprising using the catheter of claim 1.

20. The method according to claim 16 further comprising using the method to diagnose prostate cancer.

21. The method according to claim 16 further comprising using the method to monitor thermal ablation as a targeted therapy for prostate cancer.
